(19) United States
(12) Patent Application Publication
(10) Pub. No.: US 2023/0335232 A1
(43) Pub. Date: Oct. 19, 2023

(54) SYSTEM AND METHOD FOR AUTOMATED ADVERSE EVENT IDENTIFICATION

(71) Applicant: IQVIA Inc., (US)

(72) Inventors: Hui Jin, Beijing (CN); Daozhou Yao, Beijing (CN); Yubo He, Beijing City (CN); Lei Chen, Shanghai (CN); Huiying Sun, Beijing (CN); Yuan Liao, Shanghai (CN); Zhenxing Li, Beijing (CN); Yue Wang, Shanghai City (CN)

(21) Appl. No.: 17/789,345

(22) PCT Filed: Apr. 15, 2022
(86) PCT No.: PCT/CN2022/087131

§ 371 Date: Jun. 27, 2022

(51) Int. Cl. G16H 10/20; G16H 10/60; G16H 15/00

(52) U.S. Cl. CPC G16H 10/20; G16H 10/60; G16H 15/00

(57) ABSTRACT

Methods, systems, and apparatus for identifying an adverse event. In one aspect, a method includes obtaining first patient data; applying a machine learning model to the first patient data to identify information indicative of a first adverse event in the first patient data, in which the machine learning model is configured to: identify one or more named entities present in the first patient data; identify information indicative of the first adverse event based on the identified named entities; and output annotated patient data; obtaining feedback data on the annotated patient data, in which the feedback data is usable to refine the machine learning model; applying the refined machine learning model to second patient data to identify information indicative of a second adverse event in the second patient data; and providing information indicative of the second adverse events identified in the second patient data.

### TECHNICAL FIELD

The present disclosure is directed towards identifying adverse events associated with therapeutic products based on natural language processing.

### BACKGROUND

An adverse event (AE) is an event such as death, life-threatening condition, hospitalization, disability, or temporary or permanent damage, or another adverse side effect experienced by a patient receiving a particular therapeutic product as a treatment. For example, according to Federal Drug Administration (FDA) guidance for clinical trials, an adverse event represents an event, identified during ongoing monitoring of treatment of a disease using a therapeutic product in drug development, that presents a threat to patients undergoing the treatment. Identifying and reporting adverse events is a common practice during clinical trials and post-marketing pharmacovigilance.

### SUMMARY

This specification describes techniques for identifying adverse events (AEs).

In an aspect, a computer-implemented method include obtaining, by one or more processors, first patient data. The first patient data includes medical data associated with a patient who received a therapeutic product for a treatment of a disease. The method includes, by the one or more processors, applying a machine learning model to the first patient data to identify information indicative of a first adverse event in the first patient data. The machine learning model is configured to identify one or more named entities present in the first patient data, identify information indicative of the first adverse event based on the identified named entities, and output annotated patient data. The annotated patient data includes an annotation for each identified named entity in the patient data and an annotation for the information indicative of the first adverse event. The method includes, by the one or more processors, obtaining feedback data on the annotated patient data. The feedback data is usable to refine the machine learning model. The method includes, by the one or more processors, applying the refined machine learning model to second patient data to identify information indicative of a second adverse event in the second patient data. The method includes providing, by the one or more processors and for output on a user interface, information indicative of the second adverse events identified in the second patient data.

Embodiments can include one or any combination of two or more of the following features.

The medical data includes one or more of an audio-based survey, a video-based survey, and a written text.

The method includes performing natural language preprocessing. The natural language preprocessing includes one or more of transcription, translation, or decryption.

Identifying one or more named entities includes identifying each of one or more texts, in the first patient data, that match with a respective one of plurality of named entities stored in a natural language corpus.

The information indicative of the first adverse event includes one or more relationships among the named entities present in the first patient data.

The one or more relationships includes a relationship between product information and patient outcome information.

Obtaining the feedback data on the annotated patient data includes: enabling display of the annotated patient data with a plurality of annotations on the user interface; and receiving, for each annotation among the plurality of annotations, (i) an acceptance of the annotation or (ii) a rejection of the annotation.

The feedback data establishes a new relationship between the therapeutic product and a patient outcome as an adverse event.

The feedback data includes a one or more user-identified annotations identifying a named entity or an adverse event not identified by the machine learning model.

The annotated patient data includes an indication of a type of each identified named entity.

The machine learning model has been trained on a plurality of training data items, wherein each training data item includes patient data annotated with named entities and adverse events.

The method includes obtaining a data structure indicative of a regulatory requirement of adverse event reporting, wherein the data structure indicates required fields of the first and the second adverse events.

In an aspect, a system includes one or more processors and one or more storage devices storing instructions that are operable, when executed by the one or more processors, to cause the one or more processors to perform operations including obtaining, by the one or more processors, first patient data. The first patient data includes medical data associated with a patient who received a therapeutic product for a treatment of a disease. The operations includes, by the one or more processors, applying a machine learning model to the first patient data to identify information indicative of a first adverse event in the first patient data. The machine learning model is configured to identify one or more named entities present in the first patient data, identify information indicative of the first adverse event based on the identified named entities, and output annotated patient data. The annotated patient data includes an annotation for each identified named entity in the patient data and an annotation for the information indicative of the first adverse event. The operations includes, by the one or more processors, obtaining feedback data on the annotated patient data. The feedback data is usable to refine the machine learning model. The operations includes, by the one or more processors, applying the refined machine learning model to second patient data to identify information indicative of a second adverse event in the second patient data. The operations includes providing, by the one or more processors and for output on a user interface, information indicative of the second adverse events identified in the second patient data.

In an aspect, a non-transitory computer readable medium encoded with a computer program includes instructions that are operable, when executed by one or more processors, to cause the one or more processors to perform operations including obtaining, by the one or more processors, first patient data. The first patient data includes medical data associated with a patient who received a therapeutic product for a treatment of a disease. The operations includes, by the one or more processors, applying a machine learning model to the first patient data to identify information indicative of a first adverse event in the first patient data. The machine learning model is configured to identify one or more named entities present in the first patient data, identify information indicative of the first adverse event based on the identified named entities, and output annotated patient data. The annotated patient data includes an annotation for each identified named entity in the patient data and an annotation for the information indicative of the first adverse event. The operations includes, by the one or more processors, obtaining feedback data on the annotated patient data. The feedback data is usable to refine the machine learning model. The operations includes, by the one or more processors, applying the refined machine learning model to second patient data to identify information indicative of a second adverse event in the second patient data. The operations includes providing, by the one or more processors and for output on a user interface, information indicative of the second adverse events identified in the second patient data.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a block diagram of an example system for identifying adverse events (AEs).

FIG. 2 is a block diagram of an example system for training a machine learning model configured to identify named entities and AEs.

FIG. 3A-3C shows an example user interface for displaying identified named entities and AEs and obtaining user feedback.

FIG. 4 is a flowchart of example process for identifying adverse events.

FIG. 5 is a block diagram of example system components that can be used to implement a system for identifying adverse events.

### DETAILED DESCRIPTION

According to an aspect of the present disclosure, systems and methods for identifying adverse events of therapeutic products, e.g., drugs for treating diabetes, are disclosed. An adverse event (AE) is an event such as death, life-threatening condition, hospitalization, disability, or temporary or permanent damage, or another adverse side effect experienced by a patient receiving a particular therapeutic product as a treatment. For example, according to Federal Drug Administration (FDA) guidance for clinical trials, an adverse event represents an event, identified during ongoing monitoring of treatment of a disease using a therapeutic product in drug development, that presents a threat to patients undergoing the treatment. Identifying and reporting adverse events is a common practice during clinical trials and post-marketing pharmacovigilance.

The systems and methods described here relate to an approach to automated AE identification. For instance, an automated AE identification system identifies components of AEs, e.g., therapeutic product information, patient information, and patient (e.g., clinical) outcome, based on natural language processing of media, such as documents, e.g., surveys of patients by healthcare providers and reports of clinical trials. The system and methods of the present disclosure can have one or more of the following advantages. The automated approaches described here can provide computationally efficient approaches to identifying and reporting AEs, even in the face of rapidly growing AE reporting volumes. This computational efficiency is important, e.g., to comply with regulatory agency demands for complete AE reports in a timely manner (e.g., within 24 hours of occurrence of an AE).

To achieve a desirable AE identification rate (e.g., to ensure all qualifying AEs to get reported), the automated AE identification system is designed to be self-evolving, meaning that the system gradually improves itself based on feedback data. The feedback data is indicative of historical performance, e.g., AE identification rate or annotation approval rate, of a machine learning model used by the automated AE identification system. This approach enables machine learning models implemented by the AE identification systems of the present disclosure to be trained in a computationally efficient manner, e.g., deployed with a relatively small number iterations of trainings and gradually improved during use based upon the feedback data. The underlying architecture of a machine learning model used to identify AEs also enables training the AE identification system computationally efficient. In addition, the approaches described in the present disclosure leverage a machine learning model that is fine-tuned to process natural language in the healthcare domain (e.g., patient data such as electronic health records) such that named entities identified in patient data are relevant to AEs. Furthermore, the approaches described here implement a user interface that obtains feedback data from a user and provides the feedback data to a machine learning training engine for further refinement of the machine learning model.

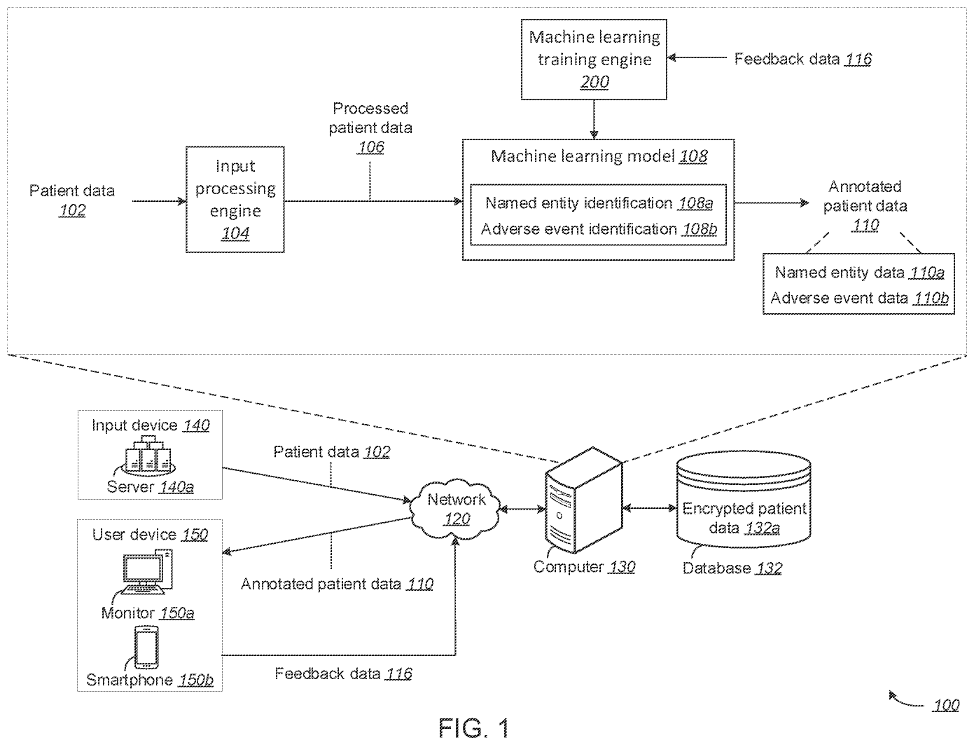

FIG. 1 is a block diagram of an example of an adverse event (AE) identification system 100 that obtains patient data 102 and generates annotated patient data 110. The system 100 includes an input device 140, a network 120, and one or more computers 130 (e.g., one or more local or cloud-based processors). The computer 130 can include an input processing engine 104, a machine learning training engine 200, and a machine learning model 108. In some implementations, the computer 130 is a server. For purposes of the present disclosure, an “engine” can include one or more software modules, one or more hardware modules, or a combination of one or more software modules and one or more hardware modules. In some implementations, one or more computers are dedicated to a particular engine. In some implementations, multiple engines can be installed and running on the same computer or computers.

The input device 140 is a device that is configured to obtain patient data 102, a device that is configured to provide patient data 102 to another device across the network 120, or any suitable combination thereof. For example, the input device 140 can include a server 140a that is configured to obtain surveys of patients for post-marketing pharmacovigilance, e.g., a recording (e.g., an audio recording or a transcription) of a conversation between a healthcare provider and a patient regarding the patient's treatment and medical concerns regarding the treatment including side effects experienced by the patient. In some implementations, the server 140a can obtain the patient data 102, e.g., by accessing medical records of a patient, and transmit the patient data 102 to another device such as the computer 130 across the network 120. In some implementations, the server 140a can obtain patient data 102 that can be accessed by one or more other input devices 140 such as computer (e.g., desktop, laptop, tablet, etc.), a smartphone, or a server. In such instances, the one or more other input devices can access the patient data 102 obtained by the server 140a and transmit the obtained patient data 102 to the computer 130 via the network 120. The network 120 can include one or more of a wired Ethernet network, a wired optical network, a wireless WiFi network, a LAN, a WAN, a Bluetooth network, a cellular network, the Internet, or other suitable network, or any combination thereof. In some implementations, the server 140a and the computer 130 are the same.

The computer 130 is configured to obtain patient data 102 from the input device 140 such as the server 140a. In some implementations, the patient data 102 can be data received over the network 120. In some implementations, the computer 130 can store the patient data 102 in a database 132 and access the database 132 to retrieve the patient data 102. The database 132, such as a local database or a cloud-based database, can store the patient data 102 that are encrypted (encrypted patient data 132a), including data for each of multiple patients, such as patient information (e.g., patient name, patient identifier, or other patient information), product information (e.g., name of drugs the patient is taking or other product information), medical history (e.g., current medical condition, past visits to doctor's office, or other medical history), AEs reported for the patient (e.g., the patient had seizure after taking drug X), or other suitable data.

The input processing engine 104 processes obtained patient data such as the patient data 102 or the encrypted patient data 132. For simplicity, we refer here to processing of patient data 102, but the description is similarly relevant to processing of the encrypted patient data 312. The patient data 102 can be text-based, non-text-based, or a combination thereof. In some implementations, the computer 130 provides the obtained patient data for input to the input processing engine 104 in the same format the patient data 102 was in when the patient data 102 was received. For example, the computer 130 can receive text-based patient data and provide the same as an input to the input processing engine 104. In some implementations, the computer 130 provides patient data in a format different from the format in which the patient data 102 was received. For instance, when the patient data is non-text-based, e.g., audio-based or video-based, the computer 130 can generate text-based patient data (e.g., a transcription) from the non-text-based patient data and provide the transcribed patient data as input to the input processing engine 104.

The input processing engine 104 is configured to receive the patient data 102 and generate processed patient data 106 for input to the machine learning model 108. The input processing engine 104 can perform natural language processing of the patient data 102, e.g., one or more of translation of the patient data 102, identification of natural language in the patient data 102, standardization of a format of the patient data 102, or other data processing. The processed patient data 106 generated by the input processing engine 104 can include a data structure that represents natural language identified in the patient data 102. The processed patient data 106 outputted by the input processing engine 104 is provided as an input to the machine learning model 108.

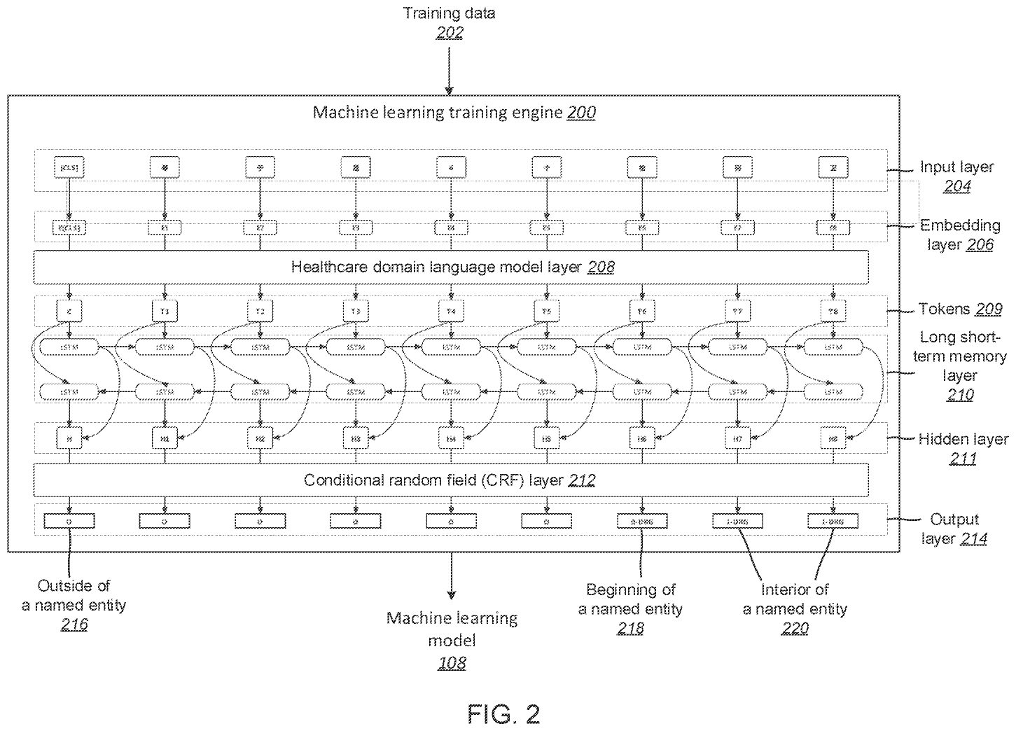

The machine learning model 108 is configured to receive the processed patient data 106, identify named entities and AEs in the processed patient data 106, and generate annotated patient data 110. The annotated patient data 110 include an annotation for each identified named entity in the processed patient data 106 (referring to as named entity data 110a) and an annotation for the information indicative of each AE identified in the processed patient data 110 (referred to as adverse event data 110b). The machine learning model 108 is a model outputted from training a machine, e.g., the computer 130; training is described in more detail referring to FIG. 2 below. The machine learning model 108 is configured to perform one or more of named entity identification 108a, adverse event identification 108b, or other appropriate processing tasks. A named entity is a phrase that identifies an item from a set of other items that have similar attributes; for example, in the sentence “Mr. X took drug Y yesterday,” Mr. X and drug Y are named entities. For named entity recognition 108a, the machine learning model 108 identifies named entities in the processed patient data 106, e.g., product information such as a drug name, patient information such as a patient name, patient outcome information such as clinical conditions experienced by a patient, prescriber information such as a prescriber name, or other named entities. For adverse event identification 108b, the machine learning model 108 identifies a set of named entities (e.g., from among those identified from the named entity identification 108a) that constitute a AE, e.g., by looking for relationships between named entities (for instance, looking for a relationship between product information and patient outcome information).

When the machine learning model 108 performs both named entity identification 108a and adverse event identification 108b, as illustrated, the machine learning model 108 uses the named entities identified during the named entity identification 108 when performing the adverse event identification 108b. In some cases, the machine learning model processed patient data 106 that already include annotations for named entities but does not itself perform named entity identification 108. In these cases, the machine learning model 108 performs the adverse event identification 108b without performing the named entity identification 108a. In some examples, the machine learning model 108 performs only named entity identification 108a but not adverse event identification 108b.

The named entity identification 108a aims to identify named entities that are relevant to AEs. For example, an AE identification criterion (e.g., as indicated by a user) can specify that each identified AE is to include certain named entities, e.g., at least product information and patient outcome information; thus, the named entity identification 108a focuses on identifying named entities relevant to product information and patient outcome information. In identifying named entities, the machine learning model 108 utilizes a natural language corpus, which is a collection of texts annotated with an entity each text belongs to: for example, “drug A” annotated with “product information” and “death” annotated with “patient outcome information”. The machine learning model 108 identifies named entities appearing in the processed patient data 106 that match, within a threshold, named entities appearing in the corpus. After identifying named entities, the machine learning model 108 generates the named entity data 110a, which is a data structure that includes an annotation for each identified named entity in the processed patient data 106. The named entity data 110a are stored in the database 132, e.g., as metadata linked to the corresponding patient data or as patient data embedded with the named entity data 110a (e.g., named entities highlighted within the patient data).

The adverse event identification 108b aims to identify relationships among the identified named entities. As described above, an AE criterion can specify that an AE is to include certain named entities. In an example, an AE is specified to include at least the product information and the patient outcome information. In this case, the adverse event identification 108b identifies relationship, in the processed patient data 106, between the product information and the patient outcome information (e.g., drug A—death). In an example, each AE is specified to include (i) the product information, (ii) the patient outcome information, and (iii) the patient information, resulting in the relationship among (i), (ii), and (iii), e.g., drug A—death—patient X. In some implementations, regulatory requirements for AE reporting defines relationships. For example, if a regulatory requirement specifies that a patient name is to be reported for each AE, the adverse event identification 108b identifies the patient name for each identified AE. In some examples, during training, the machine learning model 108 receives an input indicative of regulatory requirements, e.g., from a user operating the system. The machine learning model 108 generates adverse event data 110b, which is a data structure that includes an annotation for each identified AE in the processed patient data 106. The adverse event data 110b are stored in the database 132, e.g., as metadata linked to the corresponding patient data or as patient data embedded with the adverse event data 110b (e.g., AEs highlighted within the patient data).

As an output of applying the machine learning model 108 to the processed patient data 106, the annotated patient data 110 includes annotations for named entities (the named entity data 110a) and for AEs (the adverse event data 110b). For example, for patient data that includes the text “Patient X experienced severe headache five days after receiving drug XYZ. Patient X was hospitalized shortly after,” named entities (patient X, severe headache, drug XYZ, hospitalized) and AEs (patient X—severe headache—drug XYZ; patient X—hospitalized—drug XYZ) are annotated. In some implementations, the computer 130 generates metadata including a list of the named entities and a list of the AEs. In some implementations, the annotated patient data 110 include an annotation for dates associated with each AE, e.g., a date or time period of onset of an AE (e.g., 5 days after receiving a drug in the above example) or how long the AE persisted. In some implementations, the annotated patient data 110 includes an annotation for prescriber information.

The computer 130 can generate rendering data that, when rendered by a device having a display such as a user device 150 (e.g., a computer having a monitor 150a, a mobile computing device such as a smart phone 150b, or another suitable user device), can cause the device to output data including the annotated patient data 110. Such rendering data can be transmitted, by the computer 130, to the user device 150 through the network 120 and processed by the user device 150 or associated processor to generate output data for display on the user device 150. In some implementations, the user device 150 can be coupled to the computer 130. In such instances, the rendered data can be processed by the computer 130, and cause the computer 130, on a user interface, to output data that include the annotated patient data 110.

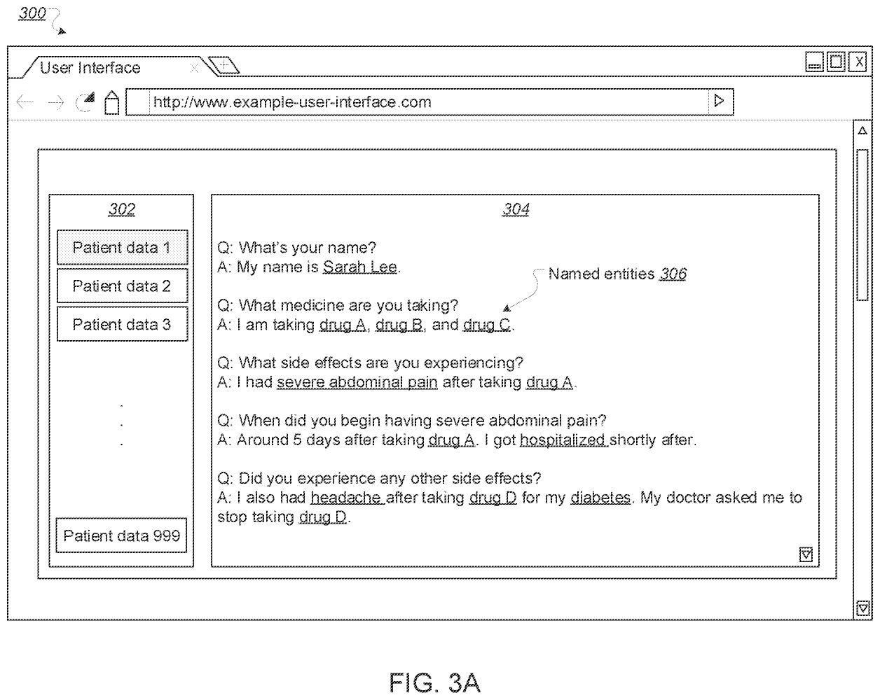

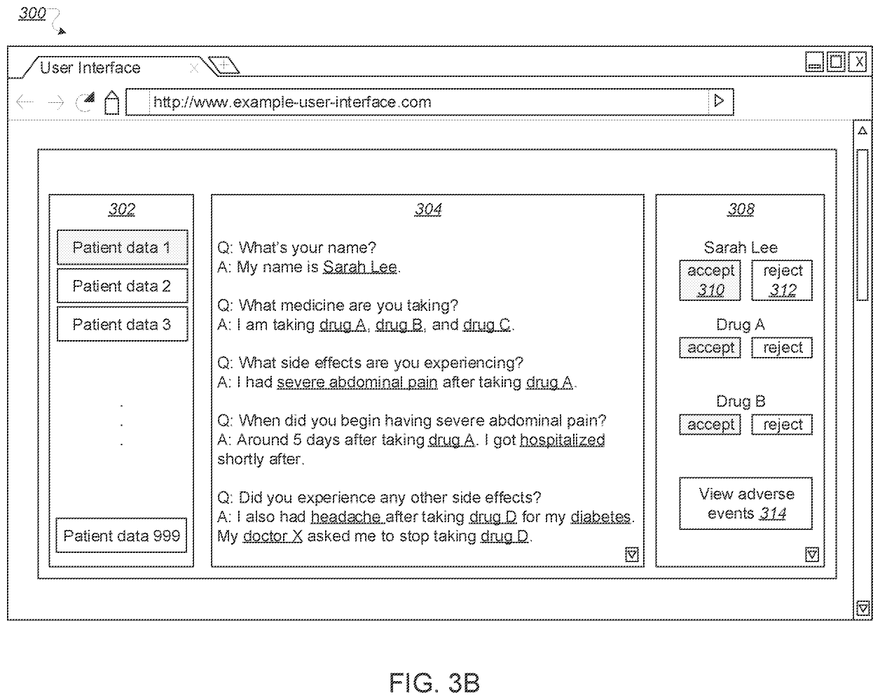

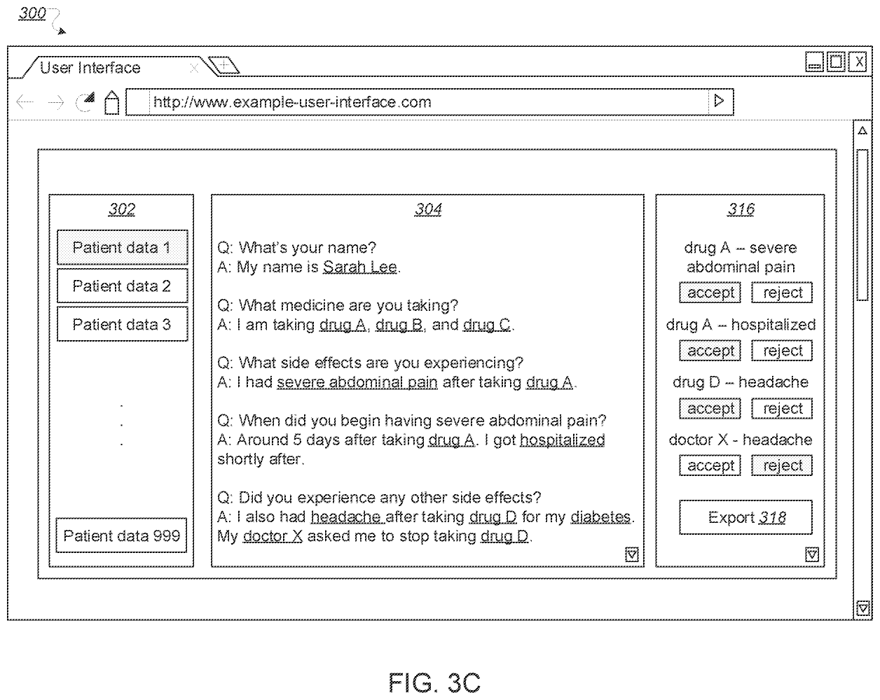

The user device 150 obtains feedback data 116 from a user, via a user interface, and provides the feedback data 116 to the computer 130 to be used by the machine learning training engine 200 for refining the machine learning model 108. The feedback data 116 represents the user's acceptance or rejection of each of named entity identified in the named entity data 110a and/or the user's acceptance or rejection of each AE identified in the adverse event data 110b. Based on the feedback data 116, the corpus is updated. For example, the user may accept an identified named entity if the named entity is relevant for AEs; the machine learning training engine 200 incorporates feedback data received from the user and updates the corpus used in the named entity identification 108a. When the user rejects an identified named entity, the machine learning training engine 200 updates the corpus to remove the rejected named entity. Similarly, the user accepts the identified AE if the identified AE is correct without missing any required field for a relationship to be formed. For the rejected AE by the user, the machine learning training engine 200 updates how it construct a relationship between components of AEs by learning features distinguishing AEs and rejected AEs. In some implementations, the feedback data 116 includes the user's manual annotation of the named entities and/or AEs in the patient data. The machine learning training engine 200 adds the manually annotated named entities to the corpus. Similarly, the machine learning training engine 200 updates the rule for the relationship based on the manually annotated AEs. The machine learning training engine 200 uses this feedback in refining the machine learning model 108, e.g., by incorporating the named entities or AEs manually identified or rejected by the user into the training. The feedback data 116 and the user interface are described in more detail, referring to FIGS. 3A-3E below.

FIG. 2 is a block diagram of an example of the machine learning training engine 200 for training or refining the training of the machine learning model 108. For example, the machine learning model can be a named entity recognition (NER) model that is trained to identify components of AEs in processed patient data 106, such as patient information (e.g., patient name, medical record number, or other patient identifier), product information (e.g., drug name), patient outcome information (e.g., side effects experienced by the patient), or other components of AEs. Upon identifying components of AEs in the processed patient data 106, the machine learning model 108 can be used to identify and annotate AEs. As an output of the machine learning training engine 200, the annotated patient data 110 is generated.

The machine learning training engine 200 obtains training data 202 and processes the training data 202. The training data 202 include patient data that have been annotated (e.g., by experts) with components of AEs present in that patient data. For example, each item of patient data is manually annotated with a patient name, a therapeutic product name, and a longitudinal patient outcome. A longitudinal patient outcome includes a date and a description of each condition (e.g., hospitalization, side effects) experienced by the patient. In the event that a patient received more than one therapeutic product, the patient outcome can be separated by the therapeutic product (e.g., when the patient began taking a new drug as of a certain date, the patient experienced severe abdominal pain). In some implementations, the training data 202 are processed using the input processing engine 104 as described referring to FIG. 1.

The machine learning model 108 uses the training data 202 to learn features distinguishing named entities from non-named entities. Specifically, the machine learning model 108 aims to identity named entities relevant to AEs (e.g., patient information, therapeutic product information, patient outcome information), not all named entities found in the patient data. Based on the identified named entities, the machine learning model 108 determines one or more AEs by looking for relationships among the identified named entities.

The machine learning training engine 200 processes the training data 202 through multiple layers. An input layer 204, which is a first layer of the multiple layers, obtains the training data 202. The training data 202 includes a set of sentences, e.g., as shown in FIG. 2, with at least some of the sentences containing named entities. “CLS” in the input layer 204 represents the start of each sentence. An embedding layer 206 generates a vector to represent each unit (e.g., character or word) of the sentence. A healthcare domain language model layer 208 obtains embeddings (annotated as “E1” to “E8”) and generates tokens 209. Each of the tokens 209 represents a word piece, such as words, phrases, characters, groups of characters, etc., that constitutes a portion of the training data. For instance: one sentence can be broken into multiple tokens (e.g., by words or phrases). For example, the example sentence shown in FIG. 2 is tokenized by word. In some implementations, the healthcare domain language model layer 208 includes pre-trained bidirectional encoder representations from transformers (BERT), a general-purpose language model. In some implementations, the healthcare domain language model layer 208 fine-tunes the BERT model by using healthcare domain training data and healthcare domain corpus. Using a healthcare domain language model rather than a general-purpose language model improves identifying named entities that are likely to be relevant to AEs.

The tokens 209 are processed by a LSTM-CRF model that includes a long short-term memory (LSTM) layer 210 and conditional random field (CRF) layer 212. The LSTM layer 210 learns long-term dependencies (e.g., such that interpretation of the sentence involves more than adjacent words) by leveraging both past and future inputs known for a period of time. As shown in FIG. 2, the LSTM layer includes multiple directional LSTMs, where each token 209 is given to forward and backward LSTMs. This enables capturing bidirectional context of the word and thus long-term dependencies. A hidden layer 211 produces a vector indicative of scores (H1 to H8), which the CRF layer 212 uses to determine a probability that each token belongs to a certain class. An output layer 214 shows the output of the CRF layer 212 for each token in the training data, where the output is a first output 216 (e.g., “0”) to indicate that the corresponding token is outside of (not part of) a named entity, a second output 218 (e.g., “B-DRG”) to indicate that the corresponding token is the beginning of a named entity, or a third output 220 (e.g., “I-DRG”) to indicate that the corresponding token is in the interior (inside) of a named entity. For instance, in the example illustrated in FIG. 2, the outputs 218-220 indicate that the last three Chinese characters in the sentence shown in the input layer 204 together correspond to a named entity appearing in the training data 202, e.g., the name of a therapeutic product. As an output of training, the machine learning training engine 200 outputs the machine learning model 108. In some implementations, the trained machine learning model 108 is saved in the database 132.

The machine learning training engine 200 can be used to refine the training of the machine learning model 108 by obtaining feedback data 116 from the user device 150 and incorporating the feedback data into training of the model 108. The feedback data 116 represents the user's acceptance or rejection of each of named entity in the named entity data 110a and/or each AE in the adverse event data 110b. The feedback data 116 can include user-identified named entities and/or AEs that were not identified by the machine learning model 108. The feedback data 116 improves the accuracy of the machine learning model 108 in one or more of the following ways: (i) updating the corpus used in the named entity identification 108a (e.g., by removing named entities not relevant to AEs and adding named entities manually annotated by the user); (ii) updating the definition of AEs (e.g., by meeting the regulatory requirements) such that there is no missing information for each AE; and (iii) including features predictive of identifying relationships among the named entities from the feedback data 116.

FIG. 3A shows an example user interface 300 for obtaining feedback data 116. The user interface 300 displays the annotated patient data and/or identified AEs and prompts a user to provide feedback on each annotation and/or each AE. The feedback data 116 represents the user's acceptance or rejection of each of the annotations and/or AEs. In some implementations, the feedback data 116 include an identification, by the user, of a named entity in the annotated patient data 110 that was not recognized by the machine learning model 108. In some implementations, the feedback data include a rejection, by the user, of term identified as an named entity in the annotated patient data 110.

In the example of FIG. 3A, the user interface 300, a web-based user interface displayed on the user device 150, displays two panels: a left panel 302 that displays available patient data and a right panel 304 that displays the selected annotated patient data (e.g., patient data 1). Annotated named entities 306 are displayed, e.g., as underlined in the right panel 304 in the patient data. In some implementations, the annotated named entities can be displayed as a list alongside of the right panel 304. In some implementations, the annotated named entities include the type each named entity belongs to; for example, patient information for “Sarah Lee”, product information for “drug A”, and patient outcome information for “hospitalized”.

Responsive to the user's selection of any one of the annotated named entities 306, the user interface 300 displays a panel 308 (shown in FIG. 3B) that prompts the user to accept or reject each annotated named entities 306. The user can accept or reject a particular identified named entity by clicking a user selectable element 310 (“accept”) or a user selectable element 312 (“reject”) respectively on the named entity. In some implementations, the user can select text that is not identified as a named entity in the panel 304 and indicate that text as a named entity.

FIG. 3C shows the user interface 300 responsive to the user's selection of a user selectable element 314 (“View adverse events”). The user can accept or reject identified AEs displayed in a panel 316. The user can select “accept” to confirm the identification of a given AE, or can select “reject” to reject an identified AE. For example, when the user determines that a particular AE is incorrect (e.g., doctor X—headache; doctor X is not the one who experienced headache), the user may reject that AE. The accepted AE can be exported in respond to the user's selection of a user selectable element 318 (“Export”) that saves AEs as a table.

The user's acceptance and rejection of named entities (through interacting with the panel 308) and AEs (through interacting with the panel 316) constitute as the feedback data 116. As described above, the machine learning training engine 200 obtains the feedback data 116 and improves the model accuracy, e.g., by updating what can be AEs and adding or deleting named entities. For example, when the user determines that a particular patient outcome such as “migraine” does not result in AEs, the machine learning training engine 200 trains the machine learning model 108 such that the machine learning model 108 does not recognize “migraine” as a named entity.

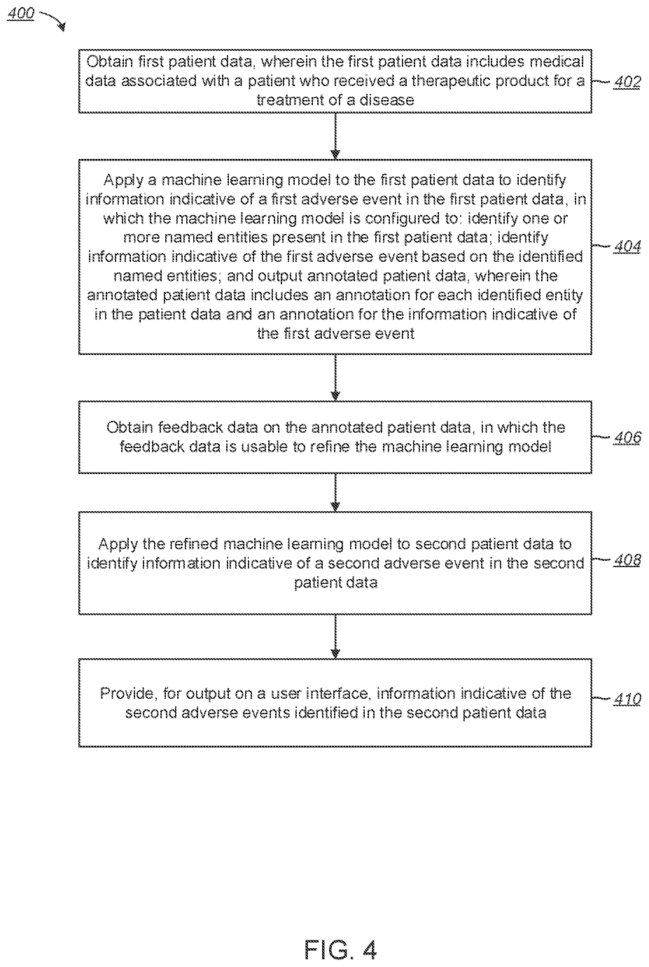

FIG. 4 is a flowchart of an example of a process 400 for identifying adverse events. The process will be described as being performed by a system of one or more computers programmed appropriately in accordance with this specification. For example, the computer 130 of FIG. 1 can perform at least a portion of the example process. In some implementations, various steps of the process 400 can be run in parallel, in combination, in loops, or in any order.

The system obtains first patient data (402). The first patient data includes medical data associated with a patient who received a therapeutic product for a treatment of a disease. The medical data includes one or more of an audio-based survey, a video-based survey, or a written text, or other suitable medical data. For example, an electronic health record of a patient is the patient data. The system can perform natural language preprocessing, including one or more of audio transcription, translation of the written text to another language, or decryption of the medical data (if the medical data is encrypted), or other suitable natural language preprocessing tasks.

The system applies a machine learning model to the first patient data to identify information indicative of a first adverse event in the first patient data (404). The machine learning model, e.g., the machine learning model 108, is configured to identify one or more named entities present in the first patient data, identify information indicative of the first adverse event based on the identified named entities, and output annotated patient data. Identifying one or more named entities includes identifying one or more texts that match with a plurality of named entities stored in a natural language corpus. For example, “brain damage”, “drug X”, and “patient Y” are example named entities appearing in the natural language corpus. The information indicative of the first adverse event includes one or more relationships (e.g., between product information and patient outcome information) among the named entities present in the first patient data. Continuing the above example, “patient Y” experienced “brain damage” after taking “drug X” is an example information indicative of the first adverse event. The annotated patient data includes an annotation for each identified entity in the patient data and an annotation for the information indicative of the first adverse event. For example, the patient data can be annotated (e.g., highlighted, underlined) for a product information (e.g., drug X), a patient outcome information (e.g., hospitalized), a provider information (e.g., physician Y, clinical trial site Z). In some implementations, the annotated patient data includes a type of each identified named entity: for example, product information for “drug X” and patient outcome information for “hospitalized.”

The machine learning model has been trained on a plurality of training data items, wherein each training data item includes patient data annotated with named entities and adverse events. In some implementations, the system obtains a data structure indicative of a regulatory requirement of adverse event reporting, the data structure that indicates criteria for identification of AEs according to the regulatory requirement (e.g., an AE is to include product information; patient information is optional).

The system obtains feedback data on the annotated patient data (406). The feedback data is usable to refine the machine learning model. The system enables display of the annotated patient data with a plurality of annotations on the user interface. For example, the user interface provides one or more user selectable elements that prompt a user whether to incorporate each annotation. The system receives, for each annotation among the plurality of annotations, (i) an acceptance of the annotation or (ii) a rejection of the annotation. In some implementations, the feedback data establishes a new relationship between named entities, e.g., between a therapeutic product and a patient outcome as an adverse event. In some implementations, the feedback data includes a plurality of user-identified annotations not identified by the machine learning model.

The system applies the refined machine learning model to second patient data to identify information indicative of a second adverse event in the second patient data (408). The machine learning model has been refined by incorporating the feedback data into the training data items. For example, the system trains the machine learning model by using the annotated patient data (after incorporating the feedback data).

The system provides, for output on a user interface, information indicative of the second adverse events identified in the second patient data (410). For example, as shown in FIG. 3C, the AEs are displayed on the user interface and can be exported as a table.

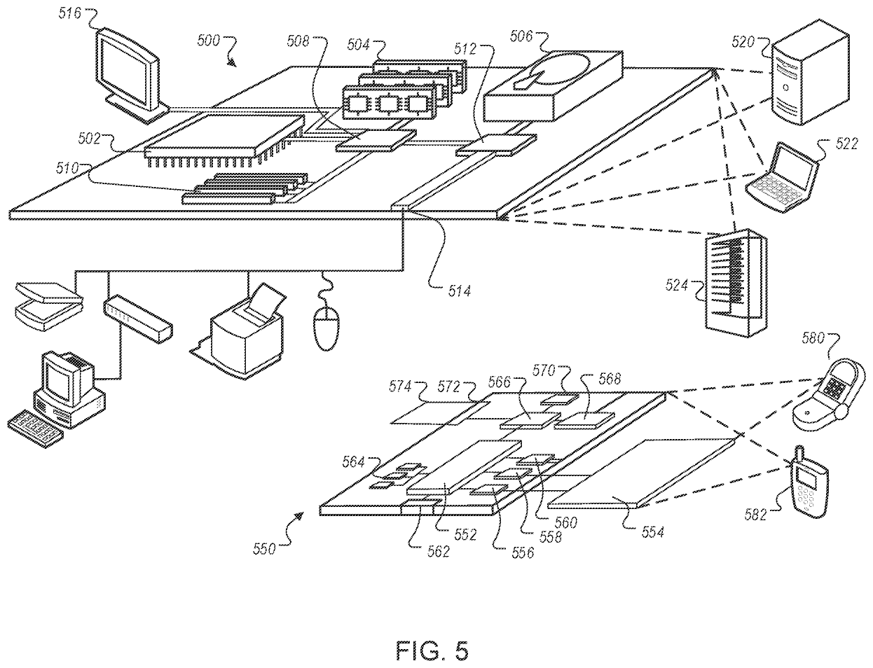

FIG. 5 is an example of a block diagram of system components that can be used to implement a system for identifying adverse events.

Computing device 500 is intended to represent various forms of digital computers, such as laptops, desktops, workstations, personal digital assistants, servers, blade servers, mainframes, and other appropriate computers. Computing device 550 is intended to represent various forms of mobile devices, such as personal digital assistants, cellular telephones, smartphones, and other similar computing devices. Additionally, computing device 500 or 550 can include Universal Serial Bus (USB) flash drives. The USB flash drives can store operating systems and other applications. The USB flash drives can include input/output components, such as a wireless transmitter or USB connector that can be inserted into a USB port of another computing device. The components shown here, their connections and relationships, and their functions, are meant to be exemplary only, and are not meant to limit implementations of the inventions described and/or claimed in this document.

Computing device 500 includes a processor 502, memory 504, a storage device 506, a high speed controller 508 connecting to memory 504 and high-speed expansion ports 510, and a low speed controller 512 connecting to low speed bus 514 and storage device 506. Each of the components 502, 504, 508, 508, 510, and 512, are interconnected using various busses, and can be mounted on a common motherboard or in other manners as appropriate. The processor 502 can process instructions for execution within the computing device 500, including instructions stored in the memory 504 or on the storage device 506 to display graphical information for a GUI on an external input/output device, such as display 516 coupled to high speed controller 508. In other implementations, multiple processors and/or multiple buses can be used, as appropriate, along with multiple memories and types of memory. Also, multiple computing devices 500 can be connected, with each device providing portions of the necessary operations, e.g., as a server bank, a group of blade servers, or a multi-processor system.

The memory 504 stores information within the computing device 500. In one implementation, the memory 504 is a volatile memory unit or units. In another implementation, the memory 504 is a non-volatile memory unit or units. The memory 504 can also be another form of computer-readable medium, such as a magnetic or optical disk.

The storage device 506 is capable of providing mass storage for the computing device 500. In one implementation, the storage device 506 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The information carrier is a computer- or machine-readable medium, such as the memory 504, the storage device 506, or memory on processor 502.

The high speed controller 508 manages bandwidth-intensive operations for the computing device 500, while the low speed controller 512 manages lower bandwidth intensive operations. Such allocation of functions is exemplary only. In one implementation, the high speed controller 508 is coupled to memory 504, display 516, e.g., through a graphics processor or accelerator, and to high-speed expansion ports 510, which can accept various expansion cards (not shown). In the implementation, low speed controller 512 is coupled to storage device 506 and low speed bus 514. The low-speed expansion port, which can include various communication ports, e.g., USB, Bluetooth, Ethernet, wireless Ethernet can be coupled to one or more input/output devices, such as a keyboard, a pointing device, microphone/speaker pair, a scanner, or a networking device such as a switch or router, e.g., through a network adapter. The computing device 500 can be implemented in a number of different forms, as shown in the figure. For example, it can be implemented as a standard server 520, or multiple times in a group of such servers. It can also be implemented as part of a rack server system 524. In addition, it can be implemented in a personal computer such as a laptop computer 522. Alternatively, components from computing device 500 can be combined with other components in a mobile device (not shown), such as device 550. Each of such devices can contain one or more of computing device 500, 550, and an entire system can be made up of multiple computing devices 500, 550 communicating with each other.

The computing device 500 can be implemented in a number of different forms, as shown in the figure. For example, it can be implemented as a standard server 520, or multiple times in a group of such servers. It can also be implemented as part of a rack server system 524. In addition, it can be implemented in a personal computer such as a laptop computer 522. Alternatively, components from computing device 500 can be combined with other components in a mobile device (not shown), such as device 550. Each of such devices can contain one or more of computing device 500, 550, and an entire system can be made up of multiple computing devices 500, 550 communicating with each other.

Computing device 550 includes a processor 552, memory 564, and an input/output device such as a display 554, a communication interface 566, and a transceiver 568, among other components. The device 550 can also be provided with a storage device, such as a micro-drive or other device, to provide additional storage. Each of the components 550, 552, 564, 554, 566, and 568, are interconnected using various buses, and several of the components can be mounted on a common motherboard or in other manners as appropriate.

The processor 552 can execute instructions within the computing device 550, including instructions stored in the memory 564. The processor can be implemented as a chipset of chips that include separate and multiple analog and digital processors. Additionally, the processor can be implemented using any of a number of architectures. For example, the processor can be a CISC (Complex Instruction Set Computers) processor, a RISC (Reduced Instruction Set Computer) processor, or a MISC (Minimal Instruction Set Computer) processor. The processor can provide, for example, for coordination of the other components of the device 550, such as control of user interfaces, applications run by device 550, and wireless communication by device 550.

Processor 552 can communicate with a user through control interface 558 and display interface 556 coupled to a display 554. The display 554 can be, for example, a TFT (Thin-Film-Transistor Liquid Crystal Display) display or an OLED (Organic Light Emitting Diode) display, or other appropriate display technology. The display interface 556 can comprise appropriate circuitry for driving the display 554 to present graphical and other information to a user. The control interface 558 can receive commands from a user and convert them for submission to the processor 552. In addition, an external interface 562 can be provide in communication with processor 552, so as to enable near area communication of device 550 with other devices. External interface 562 can provide, for example, for wired communication in some implementations, or for wireless communication in other implementations, and multiple interfaces can also be used.

The memory 564 stores information within the computing device 550. The memory 564 can be implemented as one or more of a computer-readable medium or media, a volatile memory unit or units, or a non-volatile memory unit or units. Expansion memory 574 can also be provided and connected to device 550 through expansion interface 572, which can include, for example, a SIMM (Single In Line Memory Module) card interface. The expansion memory 574 can provide extra storage space for device 550, or can also store applications or other information for device 550. Specifically, expansion memory 574 can include instructions to carry out or supplement the processes described above, and can include secure information also. Thus, for example, expansion memory 574 can be provide as a security module for device 550, and can be programmed with instructions that permit secure use of device 550. In addition, secure applications can be provided via the SIMM cards, along with additional information, such as placing identifying information on the SIMM card in a non-hackable manner.

The memory can include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 564, expansion memory 574, or memory on processor 552 that can be received, for example, over transceiver 568 or external interface 562.

Device 550 can communicate wirelessly through communication interface 566, which can include digital signal processing circuitry where necessary. Communication interface 566 can provide for communications under various modes or protocols, such as GSM voice calls, SMS, EMS, or MMS messaging, CDMA, TDMA, PDC, WCDMA, CDMA2000, or GPRS, among others. Such communication can occur, for example, through (radio-frequency) transceiver 568. In addition, short-range communication can occur, such as using a Bluetooth, Wi-Fi, or other such transceiver (not shown). In addition, GPS (Global Positioning System) receiver module 570 can provide additional navigation- and location-related wireless data to device 550, which can be used as appropriate by applications running on device 550.

Device 550 can also communicate audibly using audio codec 560, which can receive spoken information from a user and convert it to usable digital information. Audio codec 560 can likewise generate audible sound for a user, such as through a speaker, e.g., in a handset of device 550. Such sound can include sound from voice telephone calls, can include recorded sound, e.g., voice messages, music files, etc. and can also include sound generated by applications operating on device 550.

The computing device 550 can be implemented in a number of different forms, as shown in the figure. For example, it can be implemented as a cellular telephone 780. It can also be implemented as part of a smartphone 782, personal digital assistant, or other similar mobile device.

Various implementations of the systems and methods described here can be realized in digital electronic circuitry, integrated circuitry, specially designed ASICs (application specific integrated circuits), computer hardware, firmware, software, and/or combinations of such implementations. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device, e.g., magnetic discs, optical disks, memory, Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device, e.g., a CRT (cathode ray tube) or LCD (liquid crystal display) monitor for displaying information to the user and a keyboard and a pointing device, e.g., a mouse or a trackball by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The systems and techniques described here can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the systems and techniques described here, or any combination of such back end, middleware, or front end components. The components of the system can be interconnected by any form or medium of digital data communication, e.g., a communication network. Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

A number of embodiments have been described. Nevertheless, it will be understood that various modifications can be made without departing from the spirit and scope of the invention. In addition, the logic flows depicted in the figures do not require the particular order shown, or sequential order, to achieve desirable results. In addition, other steps can be provided, or steps can be eliminated, from the described flows, and other components can be added to, or removed from, the described systems. Accordingly, other embodiments are within the scope of the following claims.

1. A computer-implemented method for identifying an adverse event, the method comprising:
obtaining, by one or more processors, first patient data, wherein the first patient data includes medical data associated with a patient who received a therapeutic product for a treatment of a disease;
by the one or more processors, applying a machine learning model to the first patient data to identify information indicative of a first adverse event in the first patient data, in which the machine learning model is configured to:
identify one or more named entities present in the first patient data;
identify information indicative of the first adverse event based on the identified named entities; and
output annotated patient data, wherein the annotated patient data includes an annotation for each identified named entity in the patient data and an annotation for the information indicative of the first adverse event;
by the one or more processors, obtaining feedback data on the annotated patient data, in which the feedback data is usable to refine the machine learning model;
by the one or more processors, applying the refined machine learning model to second patient data to identify information indicative of a second adverse event in the second patient data; and
providing, by the one or more processors and for output on a user interface, information indicative of the second adverse events identified in the second patient data.

2. The computer-implemented method of claim 1, wherein the medical data comprises one or more of an audio-based survey, a video-based survey, and a written text.

3. The computer-implemented method of claim 1, further comprising:
performing natural language preprocessing, wherein the natural language preprocessing includes one or more of transcription, translation, or decryption.

4. The computer-implemented method of claim 1, wherein identifying one or more named entities comprises identifying each of one or more texts, in the first patient data, that match with a respective one of plurality of named entities stored in a natural language corpus.

5. The computer-implemented method of claim 1, wherein the information indicative of the first adverse event comprises one or more relationships among the named entities present in the first patient data.

6. The computer-implemented method of claim 5, wherein the one or more relationships comprises a relationship between product information and patient outcome information.

7. The computer-implemented method of claim 1, wherein obtaining the feedback data on the annotated patient data comprises:
enabling display of the annotated patient data with a plurality of annotations on the user interface; and
receiving, for each annotation among the plurality of annotations, (i) an acceptance of the annotation or (ii) a rejection of the annotation.

8. The computer-implemented method of claim 1, wherein the feedback data establishes a new relationship between the therapeutic product and a patient outcome as an adverse event.

9. The computer-implemented method of claim 1, wherein the feedback data comprises a one or more user-identified annotations identifying a named entity or an adverse event not identified by the machine learning model.

10. The computer-implemented method of claim 1, wherein the annotated patient data comprises an indication of a type of each identified named entity.

11. The computer-implemented method of claim 1, wherein the machine learning model has been trained on a plurality of training data items, wherein each training data item includes patient data annotated with named entities and adverse events.

12. The computer-implemented method of claim 1, further comprising:
obtaining a data structure indicative of a regulatory requirement of adverse event reporting, wherein the data structure indicates required fields of the first and the second adverse events.

13. A system comprising:
one or more processors and one or more storage devices storing instructions that are operable, when executed by the one or more processors, to cause the one or more processors to perform operations comprising:
obtaining, by the one or more processors, first patient data, wherein the first patient data includes medical data associated with a patient who received a therapeutic product for a treatment of a disease;
by the one or more processors, applying a machine learning model to the first patient data to identify information indicative of a first adverse event in the first patient data, in which the machine learning model is configured to:
identify one or more named entities present in the first patient data;
identify information indicative of the first adverse event based on the identified named entities; and
output annotated patient data, wherein the annotated patient data includes an annotation for each identified named entity in the patient data and an annotation for the information indicative of the first adverse event;
by the one or more processors, obtaining feedback data on the annotated patient data, in which the feedback data is usable to refine the machine learning model;
by the one or more processors, applying the refined machine learning model to second patient data to identify information indicative of a second adverse event in the second patient data; and
providing, by the one or more processors and for output on a user interface, information indicative of the second adverse events identified in the second patient data.

14. The system of claim 13, further comprising:
performing natural language preprocessing, wherein the natural language preprocessing includes one or more of transcription, translation, or decryption.

15. The system of claim 13, wherein identifying one or more named entities comprises identifying each of one or more texts, in the first patient data, that match with a respective one of plurality of named entities stored in a natural language corpus.

16. The system of claim 13, wherein obtaining the feedback data on the annotated patient data comprises:
enabling display of the annotated patient data with a plurality of annotations on the user interface; and
receiving, for each annotation among the plurality of annotations, (i) an acceptance of the annotation or (ii) a rejection of the annotation.

17. The system of claim 13, further comprising:
obtaining a data structure indicative of a regulatory requirement of adverse event reporting, wherein the data structure indicates required fields of the first and the second adverse events.

18. A non-transitory computer-readable medium, comprising software instructions, that when executed by a computer, cause the computer to execute operations comprising:
obtaining, by the computer, first patient data, wherein the first patient data includes medical data associated with a patient who received a therapeutic product for a treatment of a disease;
by the computer, applying a machine learning model to the first patient data to identify information indicative of a first adverse event in the first patient data, in which the machine learning model is configured to:
identify one or more named entities present in the first patient data;
identify information indicative of the first adverse event based on the identified named entities; and
output annotated patient data, wherein the annotated patient data includes an annotation for each identified named entity in the patient data and an annotation for the information indicative of the first adverse event;
by the computer, obtaining feedback data on the annotated patient data, in which the feedback data is usable to refine the machine learning model;
by the computer, applying the refined machine learning model to second patient data to identify information indicative of a second adverse event in the second patient data; and
providing, by the computer and for output on a user interface, information indicative of the second adverse events identified in the second patient data.

19. The computer-readable medium of claim 18, wherein identifying one or more named entities comprises identifying each of one or more texts, in the first patient data, that match with a respective one of plurality of named entities stored in a natural language corpus.

20. The computer-readable medium of claim 18, wherein obtaining the feedback data on the annotated patient data comprises:
enabling display of the annotated patient data with a plurality of annotations on the user interface; and
receiving, for each annotation among the plurality of annotations, (i) an acceptance of the annotation or (ii) a rejection of the annotation.
